(19) United States
(12) Patent Application Publication
(10) Pub. No.: US 2015/0342639 A1
(43) Pub. Date: Dec. 3, 2015

(54) SAFETY BUTTERFLY NEEDLE FOR TUMOR PUNCTURE

(71) Applicant: Hsien-Tsung WANG, Taipei (TW)

(72) Inventor: Hsien-Tsung WANG, Taipei (TW)

(21) Appl. No.: 14/289,665

(22) Filed: May 29, 2014

(51) Int. Cl. A61B 17/34; A61M 5/158

(52) U.S. Cl. CPC A61B 17/3496; A61M 5/158; A61M 2005/1586

(57) ABSTRACT

A safety butterfly needle for tumor puncture is provided with a needle assembly including a needle at a distal end of a needle housing, and a connector extending out of a proximal end of the needle housing; a wing having at least one winged element and a raised tunnel member for receiving the needle housing; a needle protection device including a hollow member, a slot on the hollow member and having a cavity at one end, a needle receiving cavity at an other end, and a restricting projection on the slot; and an interconnection interconnecting the hollow member and the needle housing and including a bend. The needle protection device can be pivoted toward the needle by pivoting about the bend to pass the needle through a gap between the restricting projection and the slot until being stopped by an inner surface of the hollow member.

### CROSS-REFERENCE TO RELATED APPLICATIONS

This application is a continuation in part of U.S. patent application Ser. No. 13/775,125 entitled “SAFETY BUTTERFLY NEEDLE FOR TUMOR PUNCTURE” filed Feb. 23, 2013 which is incorporated by reference for all purposes, and is now pending.

### BACKGROUND OF THE INVENTION

1. Field of the Invention

The invention relates to butterfly needles and more particularly to a safety butterfly needle for tumor puncture so that fluid can be injected into the thoracic cavity of patient.

2. Description of Related Art

It is known that butterfly needles for vein puncture are widely used in the field of medical practice. The butterfly needles are generally used for intravenous injections, infusions, blood sampling, transfusions, etc.

A typical butterfly needle comprises a needle for penetrating into the vein, a cover attached to the needle, two wings extending from both sides of the needle, a plurality of grooves formed on the cover for engaging the needle, a first slot provided on top of rear ends of the grooves, a second slot provided on bottom of rear end of the remaining grooves, and a handle extending from the cover. The wings are bent. In use, one hand holds the handle and the other hand pull the cover rearward. Further, the needle is penetrated into the vein of a patient for liquid infusion by a medical employee.

However, the typical butterfly needle for vein puncture is applicable to straight needle only not for safety butterfly needles for tumor puncture.

Safety butterfly needles for vein puncture having an L-shaped needle are applicable to patients with chronic illness. It is widely employed for liquid infusion, blood transfusion, etc. Typically, the used needles are required to conceal in order to prevent same from being used again and the sharp ends of the needles from penetrating into the skin of a medical employee due to carelessness.

Drawbacks of U.S. Pat. No. 6,921,388 include complicated structure, irregular components, uneasy manufacturing, high cost, and inconvenient operation. For example, components 50 and 74 are linked to an irregular component which is difficult of being manufactured. Further, two hinge pins 58 are pivotably secured to a hub 12, and both actuating surface 76 and edge 78 have a small operating surface, resulting in an inconvenient pressing operation of the index finger. Furthermore, components such as hub 12, actuator tab 74, mounting tube 39, holder 30, and channel 56 are complicated. In addition, hub 12, mounting tube 39, and holder 30 are secured together by glue which is not environmental friendly.

As shown in FIGS. 4, 6 and 7, after injection, a medical employee pulls needle 40 out of the chest of a patient, then snapping the needle 40 inserted into the U-shaped channel 56. The safety shield 50 will be changed an angle of about 135 degrees from an open position to a close position. However, it is not only labor intensive but also inconvenient to use due to increased operational angle. After removing the needle 40 out of the patient, a pressing of the actuating surface 76 pushes the shield 50. However, it does not insert the needle 40 into the shield 50 by snapping. Further, the U-shaped channel 56 is not disposed on the chest of the patient in a parallel relationship, i.e., at an angle of about 40 degrees with respect to the chest. Thus, the U-shaped channel 56 is subject to movement due to contact with a foreign object. This in turn can move the needle 40, thereby adversely affecting safety of blood infusion being done.

The channel 56 can be activated by pressing the actuator tab 74. While it is not shown, it can be conceived that the thumb and the middle finger are used to hold the small waist 34 or the mounting tube 39, or hold the wings 38, 36 to exert force on the actuating surface 76 of the actuator tab 74 as indicated by “A”. However, it is not a convenient operation for a medical employee.

As shown in FIGS. 4 and 6, the needle 40 is rather locked by the cannula lock 70 than in the channel 56. Thus, it is possible that the needle 40 is removed out of the channel 56, and be used again. And in turn, an individual may be pricked by the needle 40 if sufficient care is not taken when disposing.

In addition, both actuating surface 76 and edge 78 have a small operating surface, resulting in an inconvenient pressing operation of the index finger by one who is left-handed or right-handed.

The present inventor is aware that typical safety butterfly needles for vein puncture are not suitable for tumor puncture. Thus, the need for improvement still exists.

### SUMMARY OF THE INVENTION

It is therefore one object of the invention to provide a safety butterfly needle for tumor puncture comprising a needle assembly comprising a needle housing, a needle at a distal end of the needle housing, and a connector extending out of a proximal end of the needle housing; a wing comprising at least one winged element, and a raised tunnel member for releasably receiving the needle housing in friction fit; a needle protection device comprising a hollow member, a slot formed on one surface of the hollow member and including an enlarged cavity at one end, a needle receiving cavity at an other end, and at least one restricting projection horizontally extending from and disposed on a side of the slot; and an interconnection comprising an intermediate, recessed bending portion, the interconnection having one end formed integrally with the hollow member and an other end formed integrally with the needle housing; wherein the needle protection device can be pivoted toward the needle by pivoting about the bending portion to pass the needle through a gap between the restricting projection and the side of the slot until being stopped by an inner surface of the hollow member.

The invention has the following characteristics: The wing and the needle housing are manufactured separately. The wing is formed of flexible material such as thermoplastics. Thus, there is little discomfort to a patient when needle is penetrated into the vein of a patient for liquid infusion by holding the wing by a medical employee. The patient may feel a degree of comfort due to the flexibility of the thermoplastics as the material of the wing. After injection, a medical employee may pivot the hollow member to pass the needle through a gap between the first and second restricting projections by bending the first and second restricting projections. The first and second restricting projections return to its original shape after the needle passing through to dispose in the hollow member. As a result, the needle is completely concealed in the hollow member for protection. This can protect the medical employee from being pierced by the needle. Otherwise the used needle may stick the medical employee due to accidental. Further, the medical employee may dispose the safety butterfly needle after the liquid injection in order to prevent the used safety butterfly needle from being used again. Hinge pin for interconnecting needle assembly and needle protection device is not required, thereby simplifying the structure. The hinge pin is replaced with a flexible interconnection having a recessed bend. Except the wing elements all components such as needle assembly and its parts, needle protection device and its parts, and interconnection are made by injection molding in one manufacturing process. The side extensions of the needle protection device having increase area facilitate operation by a medical employee who is either right-handed or left-handed. The manufacturing cost is greatly decreased due to simplified structure and decreased number of components. Malfunction is greatly decreased. Chance of accidental prick by the needle after use is greatly decreased. No glue is used so as to be eco-friendly. Concealing the needle after use is easy and can be done in a safe manner. It is impossible of removing the concealed needle out of the needle protection device for reuse. Finally, it is very safe to use.

The above and other objects, features and advantages of the invention will become apparent from the following detailed description taken with the accompanying drawings.

### BRIEF DESCRIPTION OF THE DRAWINGS

FIG. 1 is a perspective view of a safety butterfly needle for tumor puncture according to a first preferred embodiment of the invention;

FIG. 2 is an exploded view of FIG. 1;

FIG. 3 is a side elevation in part of section of FIG. 1;

FIG. 4A is an enlarged view of the upper portion of FIG. 3;

FIG. 4B is an enlarged front view of the upper portion of FIG. 3;

FIG. 5 is a front view of FIG. 1;

FIG. 6 is a view similar to FIG. 3 showing the needle protection member being pivoted to receive the needle in use;

FIGS. 7A and 7B depict different positions of the needle and the needle protection member in use respectively;

FIGS. 8, 9 and 10 are environmental views of the safety butterfly needle for tumor puncture in use wherein FIG. 8 is shown with right forefinger being used to push;

FIG. 11 is a view similar to FIG. 8 showing left forefinger being used to push the needle into the chest of a patient;

FIG. 12 is a rear view of a safety butterfly needle for tumor puncture according to a second preferred embodiment of the invention; and

FIG. 13 is a perspective view of a safety butterfly needle for tumor puncture according to a third preferred embodiment of the invention.

### DETAILED DESCRIPTION OF THE INVENTION

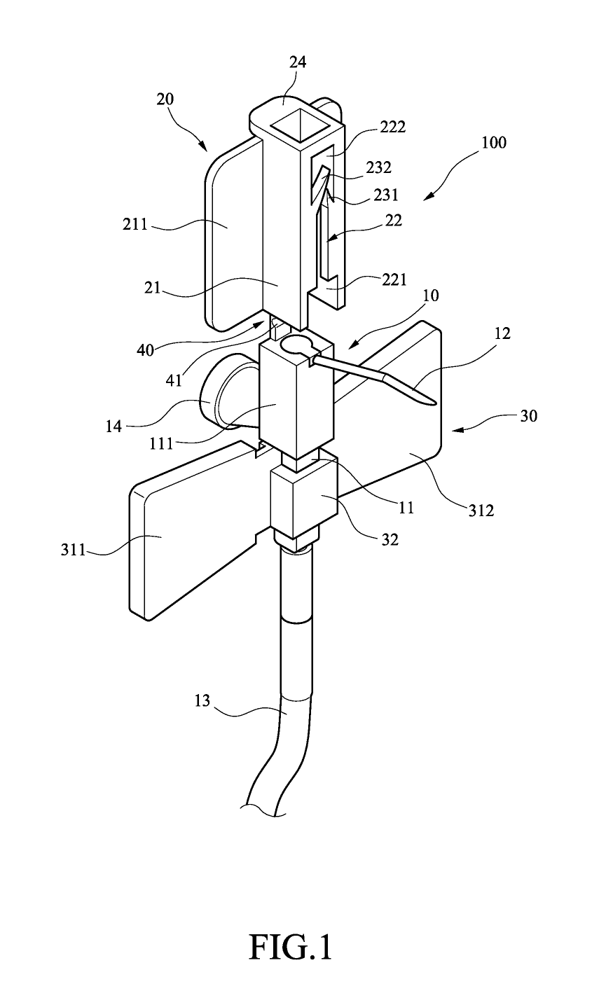

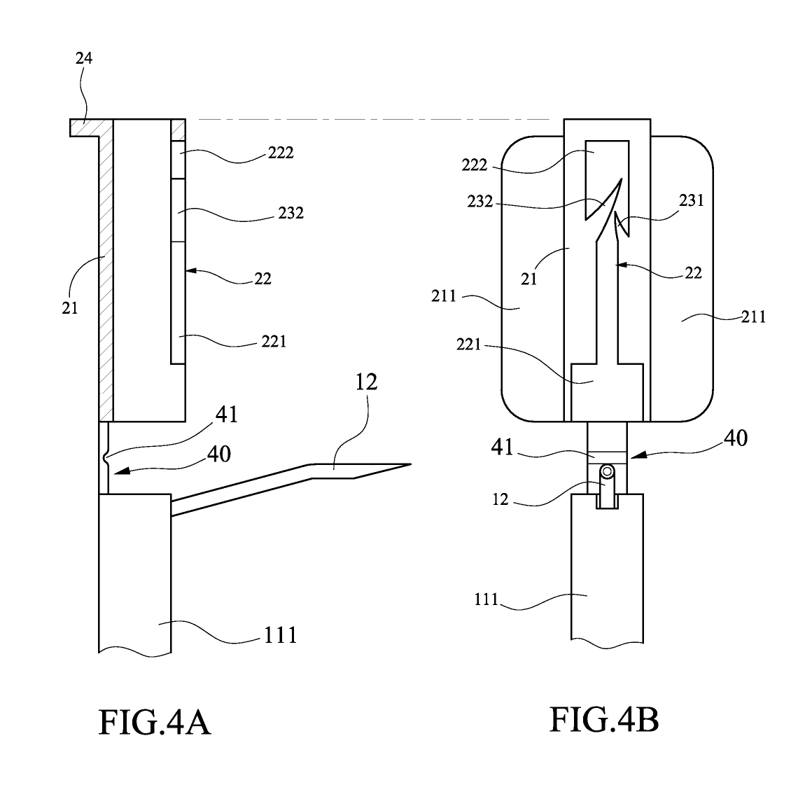

Referring to FIGS. 1 to 11, a safety butterfly needle 100 for tumor puncture in accordance with a first preferred embodiment of the invention comprises a needle assembly 10, a pivotal needle protection device 20, an interconnection 40, and a wing 30. Each component will be discussed in detail below.

The needle assembly 10 comprises a needle housing 11, a needle 12 at a distal end 111 of the needle housing 11, and a tubing 13 releasably connected to a connector 112 extending out of a proximal end of the needle housing 11. The wing 30 is put on the needle housing 11 in friction fit. The needle 12 and the needle housing 11 are generally straight. The wing 30 is of flexible material (e.g., thermoplastics) and comprises two winged elements 311, 312, and a raised tunnel member 32 for receiving the needle housing 11 in friction fit.

One drawback of the conventional safety butterfly needle is that the needle and the wings are formed integrally of hard material. This unfortunately can cause discomfort or even pain to a patient when the needle is penetrated into the vein of a patient for liquid infusion. The invention is directed to eliminate the drawback by manufacturing the wing 30 and the needle housing 11 separately in which the wing 30 is preferably formed of flexible material. Thus, there is little discomfort to a patient when the needle 12 is penetrated into the vein of a patient for liquid infusion by holding the wing 30 by a medical employee. This is one of the characteristics of the invention.

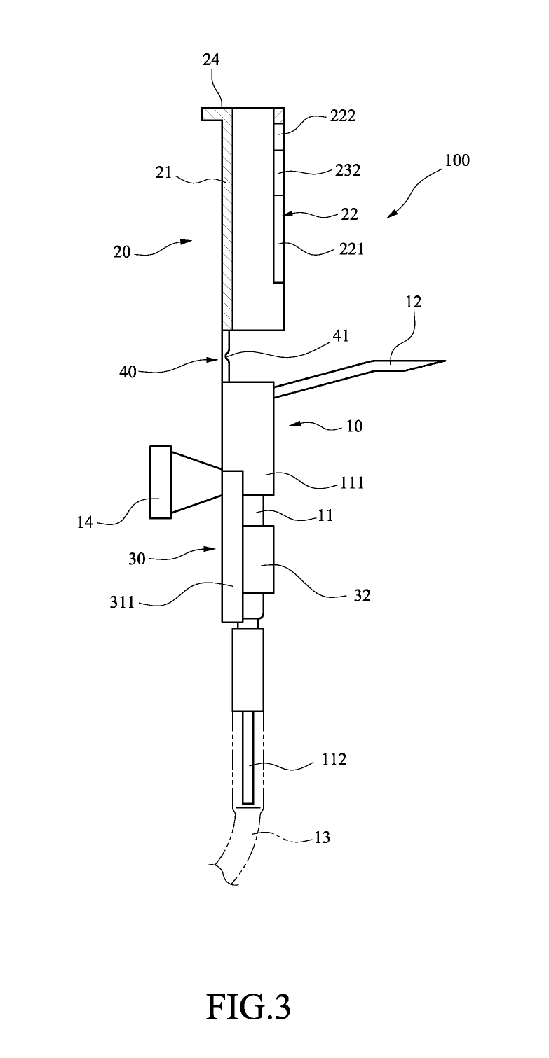

As shown in FIG. 3, a lower knob 14 is formed on a rear surface of the needle housing 11 opposite the needle 12. The knob 14 is shaped as a cone having both ends truncated as flats.

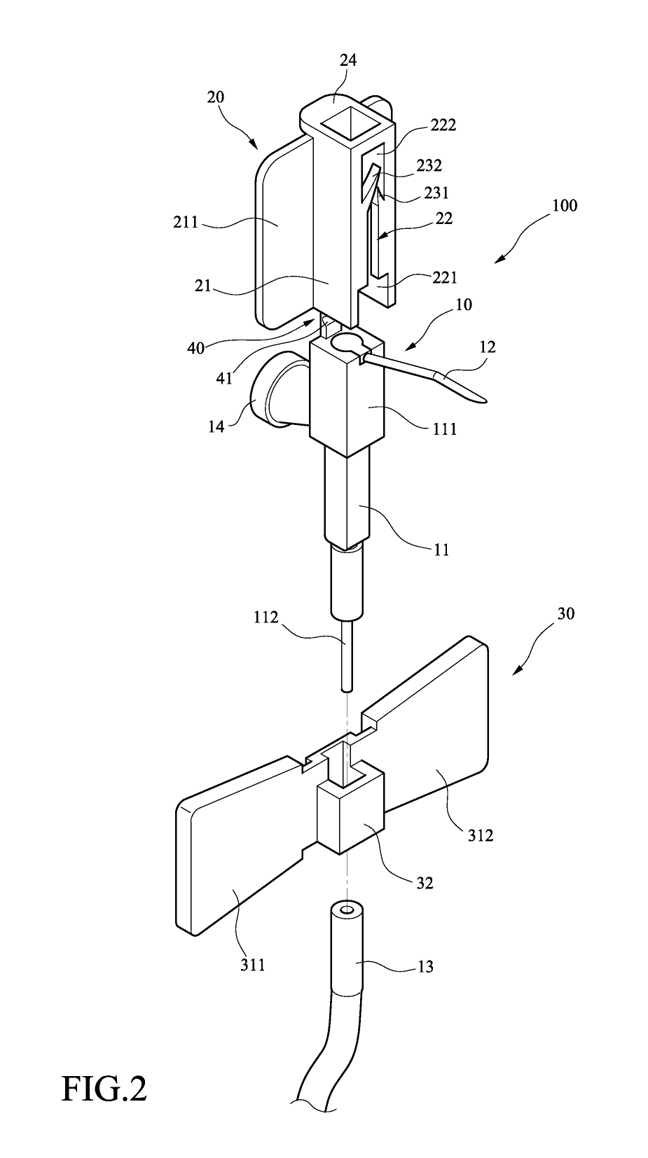

As shown in FIG. 2, the needle protection device 20 comprises a hollow member 21 of rectangular section and having two side extensions 211, and a slot 22 formed on one surface of the hollow member 21 and including an enlarged cavity 221 at a lower end and a needle receiving cavity 222 at an upper end in which the enlarged cavity 221 has a width greater than a root of the needle 12.

Opposite inclined first and second restricting projections 231, 232 are horizontally extending from and dispose formed at a joining portion of the slot 22 and the needle receiving cavity 222 with a gap formed therebetween. The needle protection device 20 further comprises an extension 24 on one top edge of the hollow member 21.

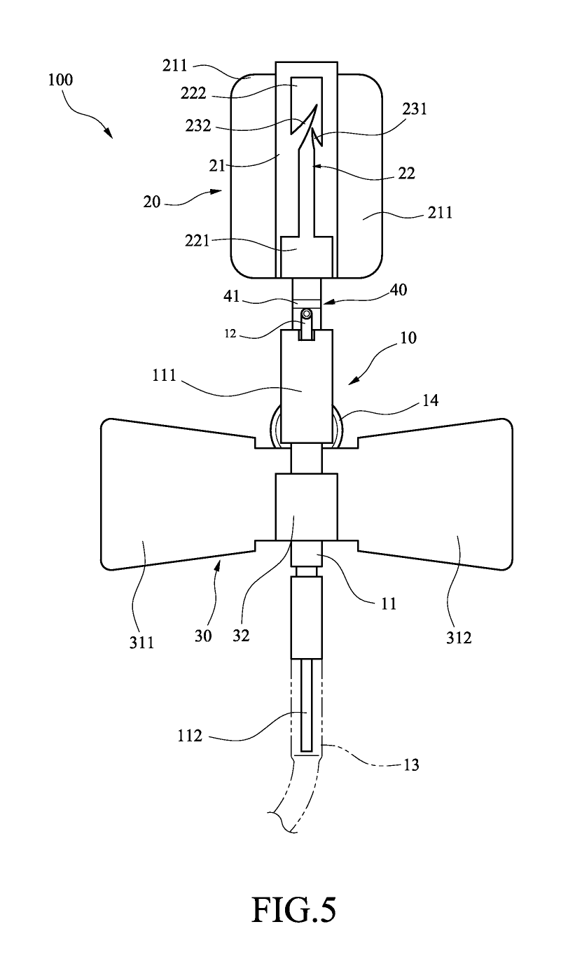

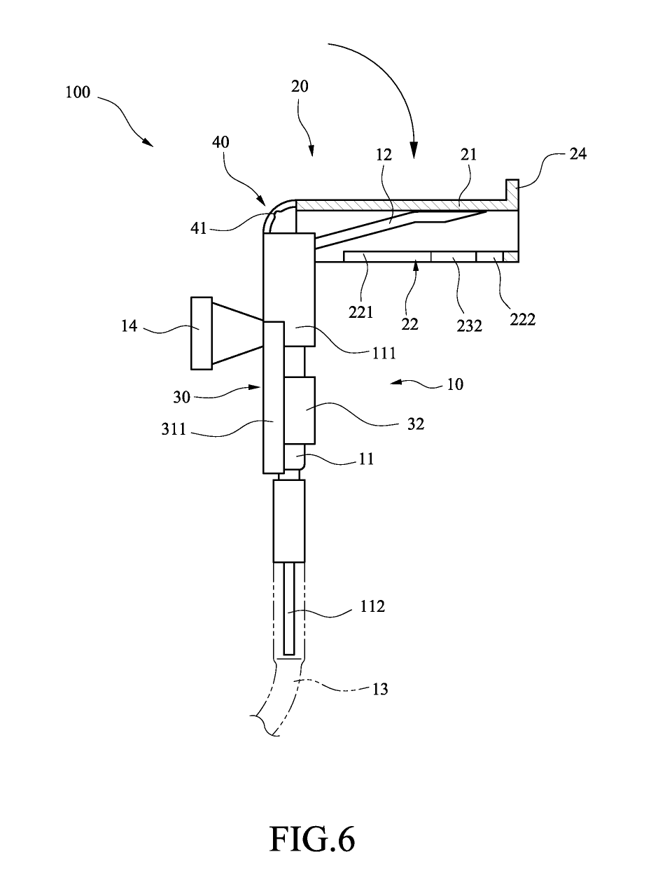

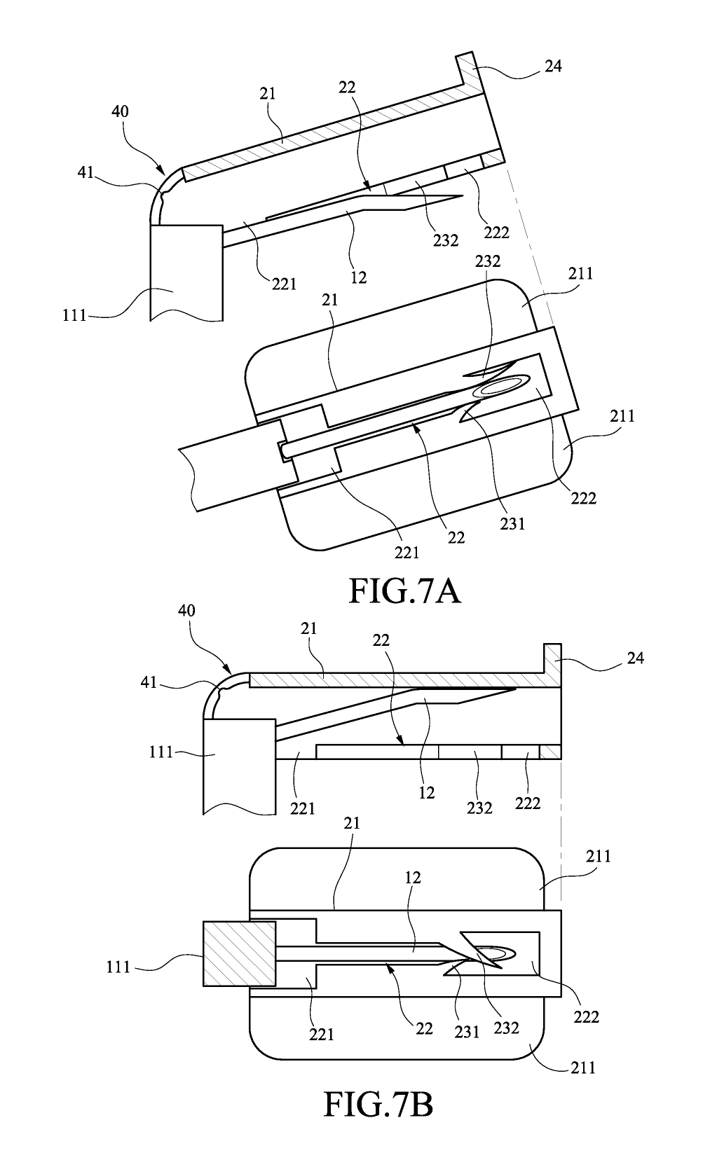

The interconnection 40 is made of flexible material and includes an intermediate, recessed bending portion 41 having a decreased thickness. The interconnection 40 has one end formed integrally with the bottom of the hollow member 21 and the other end formed integrally with the needle housing 11. The needle 12 is oriented at an angle about 90-degree with respect to the slot 22 (see FIG. 5). The space defined by the enlarged cavity 221 is sufficiently large to receive an upper portion of the needle housing 11 (i.e., the distal end 111 of the needle housing 11 joining the needle 12). As shown in FIGS. 6 to 7B, a person may pivot the needle protection device 20 by holding and pivoting the hollow member 21 toward the needle 12 about the interconnection 40. The needle 12 may pass the slot 22 and the gap between the first and second restricting projections 231, 232 until being stopped by the inner surface of the hollow member 21. The sharp end of the needle 12 is completely concealed in the hollow member 21.

It is envisaged by the invention that the first and second restricting projections 231, 232 will be bent when the needle 12 passes through the gap therebetween and return to its original shape after the needle 12 passing through. As a result, the needle 12 is completely concealed in the hollow member 21 for safety protection.

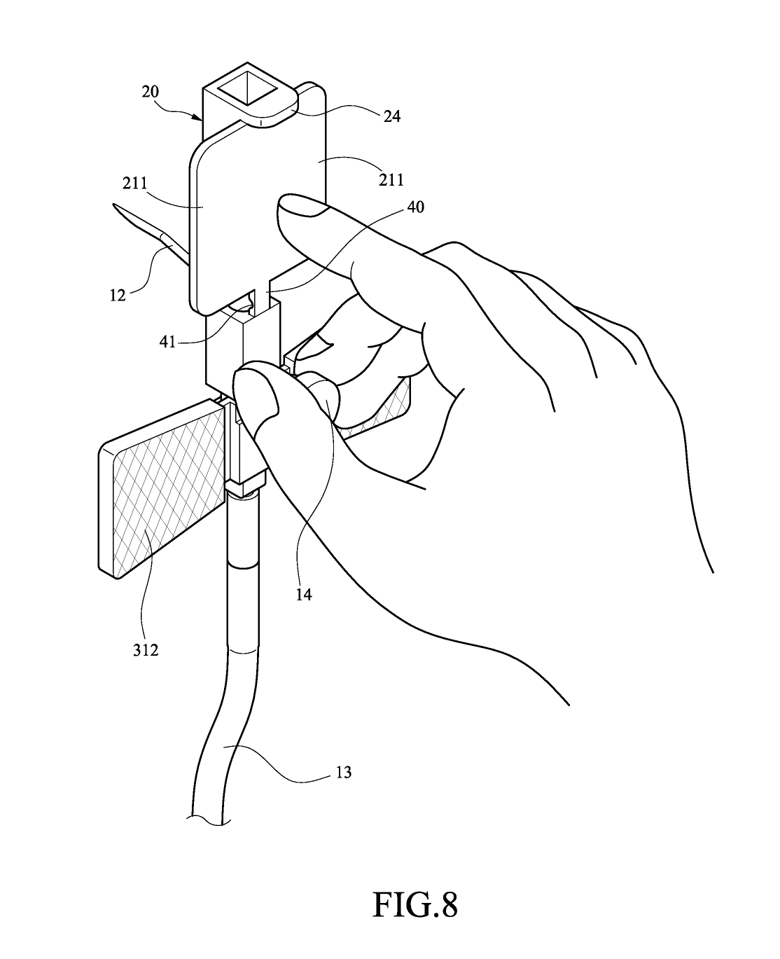

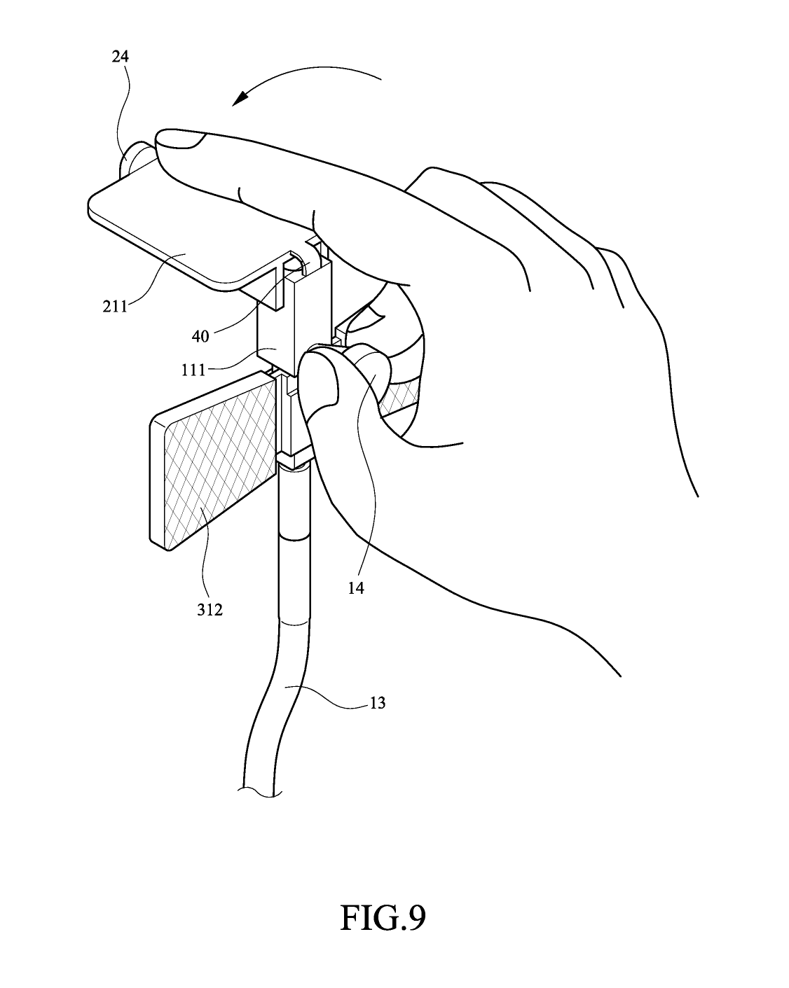

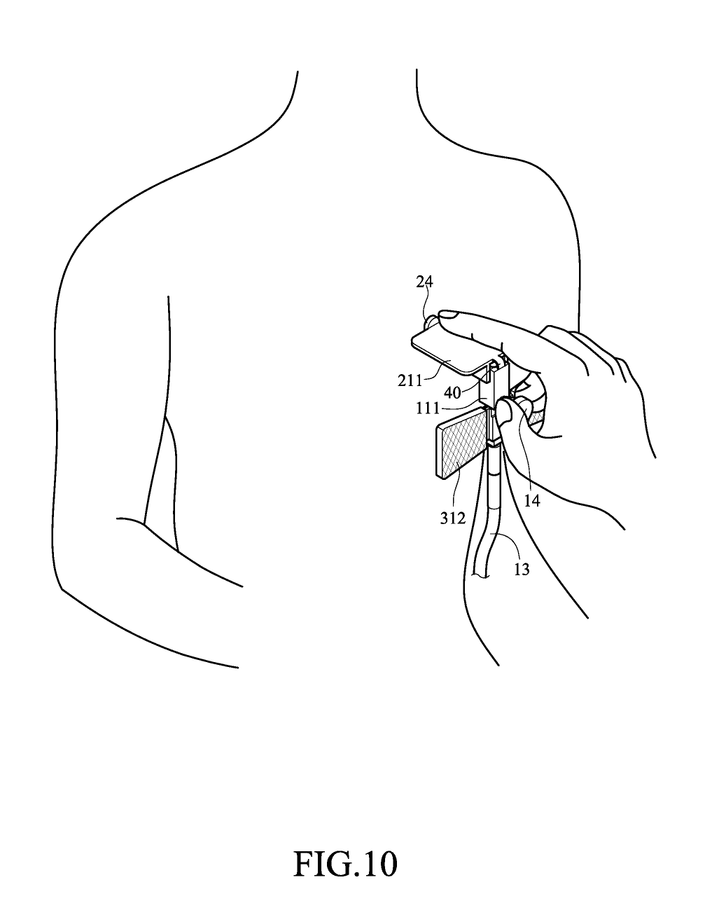

As shown in FIGS. 8, 9 and 10, use of the safety butterfly needle of the first preferred embodiment of the invention will be described in detail below. It is noted that the winged elements 311, 312 are rested upon thoracic cavity of a patient in the liquid or fluid injection process (see FIG. 10). The patient may feel a degree of comfort due to the flexibility of the thermoplastics as the material of the wing 30. While using the safety butterfly needle to inject liquid, said safety butterfly needle is flat against the patient's chest and no obstacle is outstanding, After injecting liquid into a patient's vein or thoracic cavity, a medical employee may use the thumb and the fingers other than the index finger to hold the knob 14 (FIG. 8). Next, the medical (e.g., one is right-handed) employee may further use the index finger of the right hand to pivotably press the hollow member 21 down. It is noted that a contact area of the index finger and the hollow member 21 is increased greatly due to the provision of the side extensions 211. This has the benefit of facilitating operation. Further, the hollow member 21 flexibly pivots about the needle housing 111 by bending the bending portion 41. This has the benefits of facilitating operation and decreasing the probability of malfunction. The pivotal downward movement of the hollow member 21 stops when the needle 12 is received in the needle receiving cavity 222 (FIGS. 9 and 10). This can protect the medical employee from being pierced by the needle 12. Otherwise the used needle 12 may stick the medical employee due to accident.

The medical employee may remove the safety butterfly needle 100 from the patient's chest after having completed the liquid injection process (see FIG. 10). Next, as shown in FIGS. 1 to 9, the medical employee may pivot the needle protection device 20 by holding and pivoting the hollow member 21 toward the needle 12 about the bending portion 41. The needle 12 may pass through the slot 22 and the gap between the first and second restricting projections 231, 232 until being stopped by the inner surface of the hollow member 21. The sharp end of the needle 12 is completely concealed in the hollow member 21. This can prevent the used needle 12 from being used again because the used safety butterfly needle 100 should be disposed according to regulations or order.

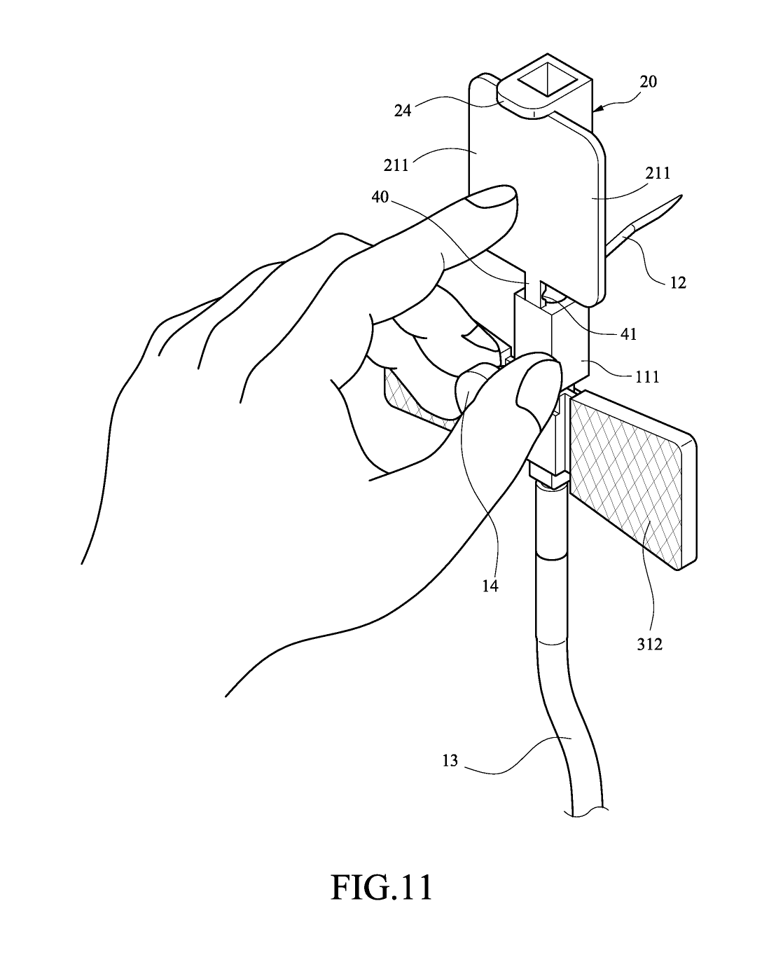

As shown in FIG. 11 specifically, use of the safety butterfly needle of the first preferred embodiment of the invention for a left-handed individual will be described in short below. After injecting liquid into a patient's vein or thoracic cavity, a medical employee may use the thumb and the fingers other than the index finger to hold the knob 14. Next, the medical employee (e.g., one is left-handed) may further use the index finger of the left hand to pivotably press the hollow member 21 down. It is noted that a contact area of the index finger and the hollow member 21 is increased greatly due to the provision of the side extensions 211. This has the benefit of facilitating operation. Further, the hollow member 21 flexibly pivots about the needle housing 111 by bending the bending portion 41. This has the benefits of facilitating operation and decreasing the probability of malfunction. The pivotal downward movement of the hollow member 21 stops when the needle 12 is received in the needle receiving cavity 222.

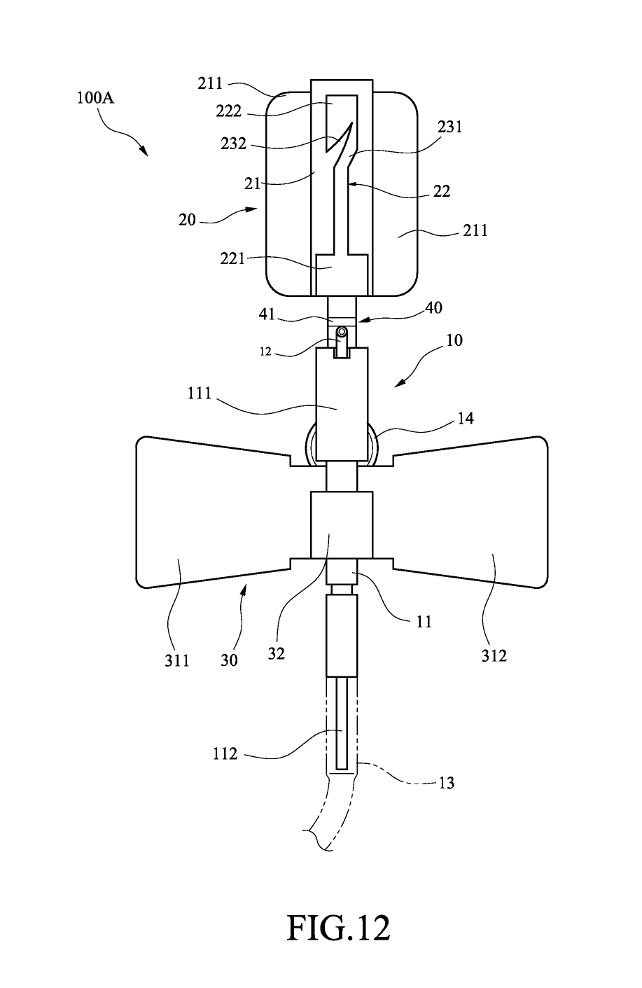

Referring to FIG. 12, a safety butterfly needle 100A for tumor puncture in accordance with a second preferred embodiment of the invention is shown. The characteristics of the second preferred embodiment are substantially the same as that of the first preferred embodiment except the following: The first restricting projection 231 is eliminated with the second restricting projection 232 remained.

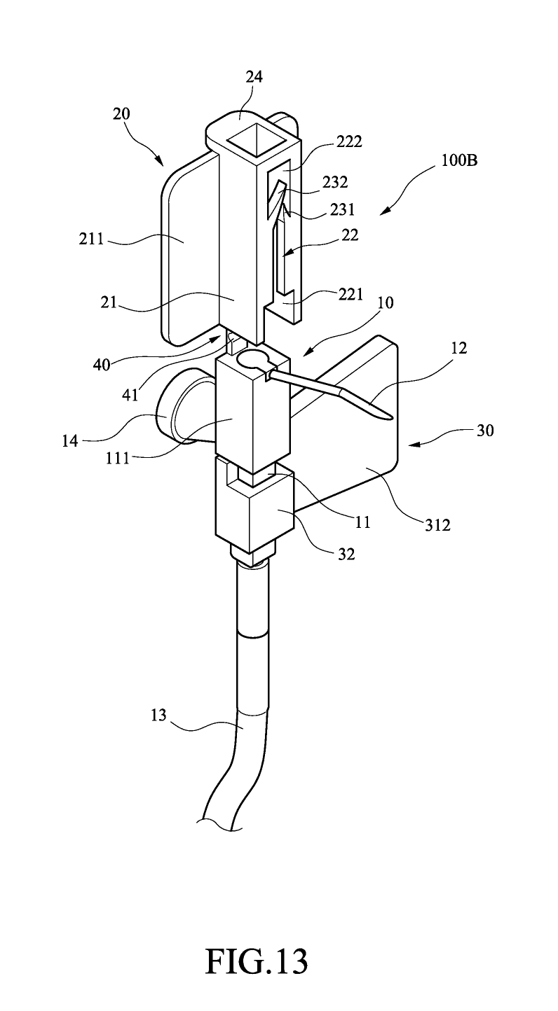

Referring to FIG. 13, a safety butterfly needle 100B for tumor puncture in accordance with a third preferred embodiment of the invention is shown. The characteristics of the third preferred embodiment are substantially the same as that of the first preferred embodiment except the following: The wing 30 has only one winged element 312 with the other winged element 311 eliminated.

While the invention has been described in terms of preferred embodiments, those skilled in the art will recognize that the invention can be practiced with modifications within the spirit and scope of the appended claims.

What is claimed is:

1. A safety butterfly needle for tumor puncture comprising:
a needle assembly comprising a needle housing, a needle at a distal end of the needle housing, and a connector extending out of a proximal end of the needle housing;
a needle protection device comprising a hollow member, a slot formed on one surface of the hollow member and including an enlarged cavity at one end, a needle receiving cavity at an other end, and at least one restricting projection horizontally extending from and disposed on a side of the slot;
a wing comprising at least one winged element, and a raised tunnel member for releasably receiving the needle housing in friction fit; and
an interconnection comprising an intermediate, recessed bending portion, the interconnection having one end formed integrally with the hollow member and an other end formed integrally with the needle housing;
wherein the needle protection device can be pivoted toward the needle by pivoting about the bending portion to pass the needle through a gap between the restricting projection and the side of the slot until being stopped by an inner surface of the hollow member.

2. The safety butterfly needle for tumor puncture of claim 1, wherein the needle assembly further comprises a knob extending from a top portion of the needle housing, the knob being spaced from the pivot and opposing the needle, and the knob being shaped as a cone having both ends truncated as flats.

3. The safety butterfly needle for tumor puncture of claim 1, wherein the needle protection device further comprises a hollow member of rectangular section and having two large side extensions each extending from the hollow member of rectangular section.

4. The safety butterfly needle for tumor puncture of claim 2, wherein the needle protection device further comprises a hollow member of rectangular section and having two large side extensions each extending from the hollow member of rectangular section.
